(19) United States
(12) Patent Application Publication
(10) Pub. No.: US 2020/0360292 A1
(43) Pub. Date: Nov. 19, 2020

(54) OIL-SOLUBLE DRUG CONTAINING COMPOSITIONS AND METHODS OF USE THEREOF

(71) Applicant: Aegis Therapeutics, LLC, San Diego, CA (US)

(72) Inventor: Edward T. Maggio, San Diego, CA (US)

(21) Appl. No.: 16/875,683

(22) Filed: May 15, 2020

Related U.S. Application Data
(60) Provisional application No. 62/849,009, filed on May 16, 2019.

(51) Int. Cl. A61K 9/48; A61K 47/44; A61K 47/10; A61K 47/26; A61K 9/00; A61K 9/107; A61K 31/047; A61K 31/352; A61K 31/192

(52) U.S. Cl. CPC A61K 9/4858; A61K 47/44; A61K 9/4825; A61K 47/10; A61K 47/26; A61K 31/7056; A61K 9/107; A61K 31/047; A61K 31/352; A61K 31/192; A61K 9/0053; A61K 9/0014

(57) ABSTRACT

The present disclosure provides compositions and formulations of cannabinoids and/or other oil-soluble drugs suitable for oral, intranasal, sublingual, transdermal and other routes of drug delivery.

### CROSS REFERENCE TO RELATED APPLICATION

This application claims the benefit of priority to U.S. Provisional Patent Application Ser. No. 62/849,009, filed on May 16, 2019. The entire contents of the foregoing is hereby incorporated by reference in its entirety.

### BACKGROUND OF THE INVENTION

### Field of the Invention

The invention relates generally to pharmaceutical formulations and more specifically to compositions and formulations of cannabinoids and/or other oil-soluble drugs which are suitable for oral, intranasal, sublingual, transdermal and other routes of administration.

### Background Information

The Cannabis plant is a source of endocannabinoids (ECBs or cannabinoids)). These are endogenous lipid-based retrograde neurotransmitters that bind to cannabinoid receptors, and cannabinoid receptor proteins that are expressed throughout the vertebrate central nervous system (including the brain) and peripheral nervous system, internal organs, connective tissues, glands, and immune cells. ECB's have a role in the pathology of many disorders and also serve a protective function in certain medical conditions. Among these, it has been proposed that migraine, fibromyalgia, irritable bowel syndrome, and related conditions represent clinical ECB deficiency syndromes (CEDS). Deficiencies in ECB signaling could be also involved in the pathogenesis of depression. In human studies, ECB system deficiencies have been implicated in schizophrenia, multiple sclerosis (MS), Huntington's disease, Parkinson's disease, anorexia, chronic motion sickness, and failure to thrive in infants. An orally administered cannabidiol solution (brand name Epidiolex®) was approved by the US Food and Drug Administration in June 2018 as a treatment for two rare forms of childhood epilepsy, Lennox-Gastaut syndrome and Dravet syndrome.

Nabiximols (brand name Sativex®), containing CBD and THC in equal proportions was approved by Health Canada in 2005 for prescription to treat central neuropathic pain in multiple sclerosis, and in 2007 for cancer related pain. In New Zealand, Sativex® is “approved for use as an add-on treatment for symptom improvement in people with moderate to severe spasticity due to multiple sclerosis.

Cannabinoids produced by the cannabis plant include, for example: THC (Δ9-tetrahydrocannabinol), CBD (Cannabidiol), CBG (Cannabigerol), CBC (Cannabichromene), CBGV (Cannabigerivarin), THCV (Tetrahydrocannabivarin), CBDV (Cannabidivarin), CBCV (Cannabichromevarin), CBGA (Cannabigerolic acid), THCA (Δ9-tetrahydrocannabinolic acid), CBDA (Cannabidiolic acid), CBCA (Cannabichromenenic acid), CBGVA (Cannabigerovarinic acid), HCVA (Tetrahydrocanabivarinic acid), CBDVA (Cannabidivarinic acid), and BCVA (Cannabichromevarinic acid).

Cannabinoid receptor type 1 (CB₁) is the most abundant G-protein-coupled receptor expressed in the central nervous system, with particularly dense expression in the sub stantia nigra, globus pallidus, hippocampus, cerebral cortex, putamen, caudate, cerebellum, and amygdala. CB₁ is also expressed in non-neuronal cells, such as adipocytes and hepatocytes, connective and musculoskeletal tissues, and the gonads. CB₂ is principally associated with cells governing immune function, although it may also be expressed in the central nervous system. THC, the major psychoactive cannabinoid is mediated by activation of the CB₁ receptors in the central nervous system. Additional ECB ligands are also known such N-arachidonyl-ethanolamide (anandamide or AEA) and sn-2-arachidonoyl-glycerol (2-AG).

Cannabidiol (CBD), a non-psychoactive cannabinoid, has been found to be useful for certain pathological conditions including pain (chronic and neuropathic), diabetes, cancer, and neurodegenerative diseases, such as Huntington's disease and exerts a therapeutic effect for epilepsy, insomnia, anxiety and/or social anxiety disorder. It has been proposed that migraine, fibromyalgia, irritable bowel syndrome, and related conditions represent clinical endocannabinoid (eCB) deficiency syndromes (CEDS). Deficiencies in eCB signaling could also be involved in the pathogenesis of depression. In human studies, eCB system deficiencies have been implicated in schizophrenia, multiple sclerosis (MS), Huntington's disease, Parkinson's disease, anorexia, chronic motion sickness, and failure to thrive in infants.

The three most common methods of administration are inhalation via smoking, inhalation via vaporization, and ingestion of edible products. The method of administration can impact the onset, intensity, and duration of psychoactive effects.

Cannabinoids are usually inhaled or taken orally. Rectal administration, sublingual administration, transdermal delivery, eye drops, and aerosols have been used in only a few studies and are of little relevance in practice today. The pharmacokinetics of THC vary as a function of its route of administration. Inhalation of THC causes a maximum plasma concentration within minutes and psychotropic effects within seconds to a few minutes. These effects reach their maximum after 15 to 30 minutes and taper off within two to three hours. Following oral ingestion, psychotropic effects manifest within 30 to 90 minutes, reach their maximum effect after two to three hours, and last for about four to 12 hours, depending on the dose. The term inhalation can be interpreted to mean an intranasal spray resulting in deposition of most or all of the formulation into the anterior portion of the nasal cavity including exposure to the olfactory bulbs and turbinates. The term inhalation is also used to mean delivery of the drugs into the lungs. This is done most often through the mouth as when smoke is drawn into the lungs during the smoking process. It is generally accepted by regulatory authorities that a spray administered into the nasal cavity having a drop size no smaller than 10 μm does not result in lung exposure and essentially all drug material is deposited in the nasal cavity alone.

Within the shifting legal landscape of medical cannabis, different methods of cannabis administration have important public health implications. A survey using data from Qualtrics® and Facebook® showed that individuals in states with medical cannabis laws had a significantly higher likelihood of ever having used the substance with a history of vaporizing marijuana and a history of oral administration of edible marijuana than those in states without such laws.

### SUMMARY OF THE INVENTION

The present invention provides therapeutic compositions and methods of use thereof to treat a subject.

Accordingly, the present disclosure provides a composition including an oil-soluble hydrophobic drug, an oil-soluble hydrophobic surfactant, and a pharmaceutically acceptable carrier, wherein the drug and the surfactant are dissolved in the pharmaceutically acceptable carrier. In embodiments, the oil-soluble hydrophobic drug is a cannabinoid and the carrier is an oil.

The present disclosure further provides a method of treating a subject which includes administering to the subject a composition of the disclosure.

### DETAILED DESCRIPTION OF THE INVENTION

Cannabinoids are frequently taken by smoking for example, or administered sublingually and subsequently swallowed, orally mixed into foods, or the rectal route. Systemic bioavailability of CBD after smoking was found to be 31+/−13%. A four-fold difference in the availability of the compound was noted between frequent and non-frequent users. Low and variable bioavailability, coupled with exposure to carcinogenic compounds resulting from inhalation of pyrolysed vegetable matter, and variability in cannabinoid content and composition in different strains or lots of cannabis plant material, make smoking marijuana both an undesirable and variable means of drug administration. Oral administration of cannabinoids, while not subject to exposure to potentially carcinogenic pyrolysis products as in smoking, is subject to the other limitations stated above, namely low and variable bioavailability, and variability in cannabinoid content and composition in different strains or lots of cannabis plant material, as well as different forms of oral composition preparation.

The present invention comprises well defined and reproducible cannabinoid compositions and formulations suitable for use in inhalation, including intranasal and lung; oral; oral cavity (buccal cell, sublingual, and laryngeal administration); dermal; topical; ocular, otic, transdermal administration, and other administration routes. Specifically, the invention comprises the use of long-chain alkylsaccharides, more specifically alkyl glycosides and alkyl esters, as non-toxic solubility and absorption enhancers, emulsifiers, and preservatives in oil-soluble drug compositions and formulations including, but not limited to, cannabinoid drugs.

The term “about,” as used herein, is intended to qualify the numerical values which it modifies, denoting such a value as variable within a range. When no range, such as a margin of error or a standard deviation to a mean value given in a chart or table of data, is recited, the term “about” should be understood to mean the greater of the range which would encompass the recited value and the range which would be included by rounding up or down to that figure as well, considering significant figures, and the range which would encompass the recited value plus or minus 20%.

The term “absorption enhancer,” as used herein, refers to a functional excipient included in formulations to improve the absorption of a pharmacologically active drug. This term usually refers to an agent whose function is to increase absorption by enhancing nasal mucous-membrane permeation, rather than increasing solubility. As such, such agents are sometimes called permeation enhancers. In particular, absorption enhancers described herein may improve paracellular transport (i.e., passage through intercellular spaces and tight junctions), transcellular transport (i.e., passive diffusion or active transport across cellular membranes), or transcytosis (i.e., cellular vesicular uptake).

Examples of absorption enhancers include aprotinin, benzalkonium chloride, benzyl alcohol, capric acid, ceramides, cetylpyridinium chloride, chitosan, cyclodextrins, deoxycholic acid, decanoyl carnitine, dodecyl maltoside, ethylenediaminetetraacetic acid (EDTA), glycocholic acid, glycodeoxycholic acid, glycofurol, glycosylated sphingosines, glycyrrhetinic acids, 2-hydroxypropyl-β-cyclodextrin, laureth-9, lauric acid, lauroyl carnitine, sodium lauryl sulfate, lysophosphatidylcholine, menthol, poloxamer 407 or F68, poly-L-arginine, polyoxyethylene-9-lauryl ether, polysorbate 80, propylene glycol, quillaia saponin, salicylic acid, sodium salt, β-sitosterol β-D-glucoside, sucrose cocoate, taurocholic acid, taurodeoxycholic acid, taurodihydrofusidic acid, and alkylsaccharides, including but not limited to dodecyl maltoside, dodecyl-β-D-maltoside, tetradecyl maltoside, tetradecyl-β-D-maltoside and sucrose dodecanoate. Alkylsaccharides (e.g., nonionic alkylsaccharide surfactants such as alkylglycosides and sucrose esters of fatty acids that consist of an aliphatic hydrocarbon chain coupled to a sugar moiety by a glycosidic or ester bond, respectively), cyclodextrins (cyclic oligosaccharides composed of six or more monosaccharide units with a central cavity, which form inclusion complexes with hydrophobic molecules and they have primarily been used to increase drug solubility and dissolution and to enhance low molecular weight drug absorption), chitosans (linear cationic polysaccharides produced from the deacetylation of chitin), and bile salts and their derivatives (such as sodium glycocholate, sodium taurocholate, and sodium taurodihydrofusidate) tend to be amongst the best-tolerated absorption enhancers.

As used herein, the term “alkylsaccharide” refers to an absorption enhancer. As used herein, an alkylsaccharide refers to any sugar joined by a linkage to any hydrophobic alkyl, as is known in the art. Alkylsaccharides can include, but are not limited to: alkylsaccharides, such as octyl-, nonyl-, decyl-, undecyk, dodecyl-, tridecyl-, tetradecyl-, pentadecyl-, hexadecyl-, heptadecyl-, and octadecyl-α- or β-D-maltoside, -glucoside or -sucroside; alkyl thiomaltosides, such as heptyl, octyl, dodecyl-, tridecyl-, and tetradecyl-β-D-thiomaltoside; alkyl thioglucosides, such as heptyl- or octyl 1-thio α- or β-D-glucopyranoside; alkyl thiosucroses; alkyl maltotriosides; long chain aliphatic carbonic acid amides of sucrose β-amino-alkyl ethers; derivatives of palatinose and isomaltamine linked by amide linkage to an alkyl chain; derivatives of isomaltamine linked by urea to an alkyl chain; long chain aliphatic carbonic acid ureides of sucrose β-amino-alkyl ethers; and long chain aliphatic carbonic acid amides of sucrose β-amino-alkyl ethers. The hydrophobic alkyl can be chosen of any desired size, depending on the hydrophobicity desired and the hydrophilicity of the saccharide moiety. For example, one preferred range of alkyl chains is from about 9 to about 24 carbon atoms. An even more preferred range is from about 9 to about 16 or about 14 carbon atoms. Similarly, some preferred saccharides include maltose, sucrose, and glucose linked by glycosidic linkage to an alkyl chain of 9, 10, 12, 13, 14, 16, 18, 20, 22, or 24 carbon atoms, e.g., nonyl-, decyl-, dodecyl- and tetradecyl sucroside, glucoside, and maltoside, etc.

As use herein, a “saccharide” is inclusive of monosaccharides, oligosaccharides or polysaccharides in straight chain or ring forms, or a combination thereof to form a saccharide chain. Oligosaccharides are saccharides having two or more monosaccharide residues. The saccharide can be chosen, for example, from any currently commercially available saccharide species or can be synthesized. Some examples of the many possible saccharides to use include glucose, maltose, maltotriose, maltotetraose, sucrose and trehalose. Preferable saccharides include maltose, sucrose and glucose.

Alkylsaccharides useful in the present invention may comprise a variety of sugars including, but not limited to, the following: maltose, maltotriose, maltotetraose sucrose, trehalose, sucrose, glucose, trehalulose, turanose, maltulose, leucrose, palatinose, isomaltose, and maltitol. Alkyl chain lengths useful in the present invention include alkyl chains ranging from eight carbons to 18 carbon. Coupling of the alkyl chain to a saccharide may be accomplished using the following types of chemical bonds: glycosidic, ester, thioglycosidic, amide, ureide, among others. Examples of such alkylsaccharides include dodecyl maltoside, octyl glucoside, tetradecyl maltoside, hexadecimal maltoside, octadecyl maltoside, decyl maltoside, sucrose dodecanoate, sucrose tetradecanoate, sucrose octadecanoate and mixed sucrose mono and dioctadecanoate.

Certain alkylsaccharides are soluble in oils and water, particularly those with alky chain lengths of 10 or more carbons. Alkylsaccharides with carbon chain lengths greater than 14 carbons are poorly soluble in water but very soluble in oils. Cannabinoids are hydrophobic and can be dissolve in organic solvents including pharmaceutically acceptable oils or may be prepared as aqueous emulsions for which alkylsaccharides may be the primary emulsifying surfactant or may be added to an emulsion.

The term “pharmaceutically acceptable,” as used herein, refers to a component of a pharmaceutical formulation that is compatible with the other ingredients of the formulation and not overly deleterious to the recipient thereof. In embodiments, a pharmaceutically acceptable carrier of the invention includes an oil or surfactant.

Pharmaceutically acceptable oils for use in the invention may include Vitamin E comprising one or more natural or synthetic tocopherols or tocotrienols selected from the group consisting of: α-tocopherol, β-tocopherol, γ-tocopherol, δ-tocopherol, α-tocotrienol, β-tocotrienol, γ-tocotrienol, δ-tocotrienol, tocophersolan, any isomers thereof, any esters thereof, any analogs or derivatives thereof, and any combinations thereof. Emulsion forming surfactants include the alkylsaccharides listed above along with others such as sodium lauryl sulfate, ammonium laureth sulfate (disodium lauryl sulfosuccinate, cocoamphocarboxyglycinate, decyl polygluco side, cetearyl alcohol, stearyl alcohol, cocamidopropyl betaine, decyl glucoside, glyceryl cocoate, sodium cocoyl isethionate, almond glyceride, sodium lauryl sulphoacetate, sodium lauroyl sarcosinate, sodium methyl cocoyl taurate, sucrose cocoate, polysorbate 20, polysorbate 80, among others.

Additional examples of pharmaceutically acceptable oils for use in the invention include oils derived from plants (e.g., corn oil, coconut oil, linseed oil, olive oil, palm oil, sunflower seed oil, safflower oil, cottonseed oil, peanut oil, sesame oil, or castor oil, canola oil, rice bran oil, soybean oil, almond oil), animals (e.g., sardine oil, or cod-liver oil) or triglyceride derivatives such as miglyol (Lab afac, Gattefusse, Lyon, France), or mixtures thereof. Further examples of pharmaceutically acceptable oils for use in the invention include cashew oil, hazelnut oil, macadamia oil, pecan oil, pistachio oil and walnut oil. Other examples of pharmaceutically acceptable oils for use in the invention include ethyl butyrate, ethyl caprylate, ethyl oleate and the triglycerides.

In certain aspects, the oil is a triglyceride, triethyl, triester or combination thereof. In embodiments, the oil is triacetin, triethyl citrate, or combination thereof.

In some aspects, the triglyceride is caproic acid, caprylic acid, capric acid, lauric acid, myristic acid or any combination thereof. For example, the triglyceride is a medium chain triglyceride, such as caprylic/capric (C8 and/or C10) triglycerides or caprylic (C8) triglycerides. In one aspect, the triglyceride may be a fatty acid ester emollient, such as a saturated coconut and palm kernel oil-derived caprylic/capric fatty acid mixture with glycerin in a solid form sold under the trademark MIGLYOL™. In another aspect, the triglyceride may be a fatty acid ester emollient, such as a saturated coconut and palm kernel oil-derived caprylic/capric fatty acid mixture sold under the trademark CAPTEX™, such as CAPTEX™ 8000.

In certain aspects, the oil pharmaceutically acceptable oil may also contain one or more pharmaceutically acceptable liquid carriers, e.g., solvents of the drug or other components of the formulation such as ethanol. The oil vehicle may also include a pharmaceutically acceptable surfactant. Examples of suitable surfactants include polyglycosides such as dodecyl maltoside or tetradecylmaltoside.

In certain embodiments, the surfactant is chosen from benzalkonium chloride, methylparaben, sodium benzoate, benzoic acid, phenyl ethyl alcohol, and the like, and mixtures thereof; surfactants such as Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and the like, and mixtures thereof.

In certain embodiments, the formulation additionally comprises a stabilizing agent.

In certain embodiments, the stabilizing agent is EDTA.

In some embodiments the composition has a pH of about 3, about 3.5, about 4, about 4.5, about 5, about 5.5, about 6, about 6.5, about 7, or about 7.5.

In some embodiments, the absorption enhancer is an alkylsaccharide, for example, a nonionic alkylsaccharide surfactant such as an alkylglycoside or a sucrose ester of fatty acids that consists of an aliphatic hydrocarbon chain coupled to a sugar moiety by a glycosidic or ester bond, respectively. In some embodiments, the absorption enhancer is an alkylmaltoside (e.g., a tetradecyl maltoside (TDM), a dodecyl maltoside, etc.). In some embodiments, the absorption enhancer is sucrose dodecanoate. Alkylsaccharides are used in commercial food and personal care products and have been designated Generally Recognized as Safe (GRAS) substances for food applications. They are non-irritating enhancers of transmucosal absorption that are odorless, tasteless, non-toxic, non-mutagenic, and non-sensitizing in the Draize test up to a 25% concentration. Without being bound to any theory, it is believed that alkylsaccharides increase absorption by increasing paracellular permeability, as indicated by a decrease in transepithelial electrical resistance; they may also increase transcytosis. The effect may be short-lived.

In some embodiments, the absorption enhancer is Intravail®, the alkylsaccharide 1-O-n-dodecyl-β-D-maltopyranoside (alternately referred to as lauryl-β-D-maltopyranoside, dodecyl maltopyranoside, dodecyl maltoside, and DDM; C₂₄H₄₆O₁₁). In certain embodiments, an intranasal formulation comprises about 0.01% to about 2.5% Intravail®. In certain embodiments, an intranasal formulation comprises about 0.1% to about 0.5% Intravail®. In certain embodiments, an intranasal formulation comprises about 0.15% to about 0.35% Intravail®. In certain embodiments, an intranasal formulation comprises about 0.15% to about 0.2% Intravail®. In certain embodiments, an intranasal formulation comprises about 0.18% Intravail®. In certain embodiments, an intranasal formulation comprises about 0.2% to about 0.3% Intravail®. In certain embodiments, an intranasal formulation comprises about 0.25% Intravail®.

An oil-soluble hydrophobic drug for use in the composition of the invention is not particularly limited, as long as it is a pharmaceutically active agent that can be dissolved in oil. Moreover, an amphipathic drug that is soluble in oil may also be used.

Accordingly, the disclosure provides a pharmaceutical composition including at least one oil-soluble hydrophobic drug in an amount effective for treating a disease or disorder, and a pharmaceutically acceptable carrier optionally including a surfactant, such as an oil-soluble hydrophobic surfactant. It is noted that the pharmaceutically active agent noted in any of the compositions herein may be substituted or augmented with a large variety of pharmaceutically active agents. Further, a pharmaceutically active agent may be hydrated; e.g., a monohydrate or dihydrate form of the molecule.

A suitable pharmaceutically active agent for use in the compositions described herein is an active pharmaceutical agent or a combination of a plurality of active agents. Such active pharmaceutical agents include, by way of illustration only, cannabinoids, anthelmintics, analgesics, antiemetics, anti-inflammatories, steroids, sedatives, antimicrobials, stimulants, antidepressants, opioids, opiates, NSAIDS, and anesthetics.

In certain aspects, the active pharmaceutical agent is an antimicrobial agent, such as an antiviral agent, antibiotic agent or antifungal agent. As such, the disclosure provides compositions comprising at least one antiviral agent, antimicrobial agent or antifungal agent in an amount effective for treating a disease or disorder, such as a microbial infection, and a pharmaceutically acceptable vehicle.

In one aspect, the active pharmaceutical agent is an antiviral agent. Examples of antiviral agents include nucleoside analogs, such as, but not limited to remdesivir, abacavir, acyclovir, adefovir, brivudine, cidofovir, clevudine, didano sine, edoxudine, emtricitabine, entecavir, famciclovir, floxuridine, ganciclovir, idoxuridine, inosine pranobex, lamivudine, penciclovir, sorivudine, stavudine, ribavirin, telbivudine, tenofovir, trifluridine, valacyclovir, valganciclovir, vidarabine, zalcitabine, and zidovudine. In certain aspects, the antiviral agent is one that is effective in treating or preventing an infectious diseases such as those caused by Ebola virus, Zika virus, influenza or coronaviruses such as Coronavirus Disease 2019 (COVID-19), SARS associated coronavirus (SARS-CoV), or Middle East respiratory syndrome coronavirus (MERS-CoV).

In a certain aspect, the antiviral agent is remdesivir. Accordingly, the disclosure provides a method of treating an infectious disease in a subject by administering a composition of the invention which includes remdesivir, wherein the subject is infected with a virus, such as Ebola virus, Zika virus, influenza or a coronavirus, such as Coronavirus Disease 2019 (COVID-19), SARS associated coronavirus (SARS-CoV), or Middle East respiratory syndrome coronavirus (MERS-CoV).

It will be appreciated that a composition of the invention including an antiviral agent may be administered to the subject via any number of administration routes. In one aspect, the composition is administered to the subject nasally as a nasally formulated composition and the antiviral agent is a nucleoside analog, such as remdesivir.

In some embodiments, there are provided (non-aqueous) pharmaceutical solutions for nasal administration comprising: (a) an antiviral agent, such as remdesivir; (b) one or more natural or synthetic tocopherols or tocotrienols, or any combinations thereof, in an amount from about 30% to about 95% (w/w); (c) one or more alcohols or glycols, or any combinations thereof, in an amount from about 10% to about 70% (w/w); and (d) an alkyl glycoside, in a pharmaceutically-acceptable solution for administration to one or more nasal mucosal membranes of a patient. In some embodiments, the antiviral agent is dissolved in the one or more natural or synthetic tocopherols or tocotrienols, or any combinations thereof, in an amount from about 30% to about 95% (w/w); and the one or more alcohols or glycols, or any combinations thereof, in an amount from about 10% to about 70% (w/w). In some embodiments, the antiviral agent is selected from the group consisting of: remdesivir, abacavir, acyclovir, adefovir, brivudine, cidofovir, clevudine, didanosine, edoxudine, emtricitabine, entecavir, famciclovir, floxuridine, ganciclovir, idoxuridine, inosine pranobex, lamivudine, penciclovir, sorivudine, stavudine, ribavirin, telbivudine, tenofovir, trifluridine, valacyclovir, valganciclovir, vidarabine, zalcitabine, and zidovudine.

In some embodiments, the solution consists of antiviral agent, such as remdesivir, alkyl glycoside (0.01-1% (w/v)), vitamin E (45-65% (w/v)), ethanol (10-25% (w/v)) and benzyl alcohol (5-15% (w/v)). In some embodiments, the solution comprises at least about 0.01% (w/w) of an alkyl glycoside, e.g. about 0.01% to 3% (w/w) of an alkyl glycoside, such as dodecyl maltoside. In some embodiments, the solution consists of antiviral agent, dodecyl maltoside (0.01-5% (w/v)), vitamin E (45-65% (w/v)), ethanol (10-25% (w/v)) and benzyl alcohol (5-15% (w/v)); more particularly the solution may consist of antiviral agent, dodecyl maltoside (0.1-2.5% (w/v)), vitamin E (50-60% (w/v)), ethanol (15-22.5% (w/v)) and benzyl alcohol (7.5-12.5% (w/v)); and even more particularly, the solution may consist of antiviral agent, dodecyl maltoside (0.15-0.5% (w/v)), vitamin E (50-60% (w/v)), ethanol (17-20% (w/v)) and benzyl alcohol (10-12% (w/v)).

In certain aspects, the active pharmaceutical agent is a selective calcitonin gene-related peptide (CGRP) antagonist. As such, the disclosure provides compositions comprising a CGRP antagonist in an amount effective for treating a disease or disorder, such as a migraine headache, and a pharmaceutically acceptable vehicle.

In some embodiments of the invention, the CGRP antagonist is ubrogepant (Ubrelvy™), a medication used for the acute (immediate) treatment of migraine with or without aura, rimegepant (Nurtec™), or atogepant. However, it will be understood that the invention is not limited to these small molecule calcitonin gene-related peptide (CGRP) antagonist.

In some embodiments, there are provided (non-aqueous) pharmaceutical solutions for nasal administration comprising: (a) a CGRP antagonist; (b) one or more natural or synthetic tocopherols or tocotrienols, or any combinations thereof, in an amount from about 30% to about 95% (w/w); (c) one or more alcohols or glycols, or any combinations thereof, in an amount from about 10% to about 70% (w/w); and (d) an alkyl glycoside, in a pharmaceutically-acceptable solution for administration to one or more nasal mucosal membranes of a patient. In some embodiments, the CGRP antagonist is dissolved in the one or more natural or synthetic tocopherols or tocotrienols, or any combinations thereof, in an amount from about 30% to about 95% (w/w); and the one or more alcohols or glycols, or any combinations thereof, in an amount from about 10% to about 70% (w/w).

In some embodiments, the solution consists of a CGRP antagonist, alkyl glycoside (0.01-1% (w/v)), vitamin E (45-65% (w/v)), ethanol (10-25% (w/v)) and benzyl alcohol (5-15% (w/v)). In some embodiments, the solution comprises at least about 0.01% (w/w) of an alkyl glycoside, e.g. about 0.01% to 3% (w/w) of an alkyl glycoside, such as dodecyl maltoside. In some embodiments, the solution consists of a CGRP antagonist, dodecyl maltoside (0.01-5% (w/v)), vitamin E (45-65% (w/v)), ethanol (10-25% (w/v)) and benzyl alcohol (5-15% (w/v)); more particularly the solution may consist of a CGRP antagonist, dodecyl maltoside (0.1-2.5% (w/v)), vitamin E (50-60% (w/v)), ethanol (15-22.5% (w/v)) and benzyl alcohol (7.5-12.5% (w/v)); and even more particularly, the solution may consist of a CGRP antagonist, dodecyl maltoside (0.15-0.5% (w/v)), vitamin E (50-60% (w/v)), ethanol (17-20% (w/v)) and benzyl alcohol (10-12% (w/v)).

The disclosure further provides compositions which include at least one anti-inflammatory agent in an amount effective for treating a disease or disorder, such as pain or inflammation, and a pharmaceutically acceptable vehicle.

In one aspect, an anti-inflammatory agent for use in a composition described herein is an NSAID. As used herein, the term “NSAID” refers to a class of therapeutic compounds with analgesic, anti-inflammatory, and anti-pyretic properties. NSAIDs reduce inflammation by blocking cyclooxygenase. NSAIDs may be classified based on their chemical structure or mechanism of action. Non-limiting examples of NSAIDs include a salicylate derivative NSAID, a p-amino phenol derivative NSAID, a propionic acid derivative NSAID, an acetic acid derivative NSAID, an enolic acid derivative NSAID, a fenamic acid derivative NSAID, a non-selective cyclooxygenase (COX) inhibitor, a selective cyclooxygenase 1 (COX-1) inhibitor, and a selective cyclooxygenase 2 (COX-2) inhibitor. A NSAID may be a profen. Examples of a suitable salicylate derivative NSAID include, without limitation, Acetylsalicylic acid (aspirin), Diflunisal, Hydroxylethyl Salicylate, and Salsalate. Examples of a suitable p-amino phenol derivative NSAID include, without limitation, Paracetamol and Phenacetin. Examples of a suitable propionic acid derivative NSAID include, without limitation, Alminoprofen, Benoxaprofen, Dexketoprofen, Fenoprofen, Flurbiprofen, Ibuprofen, Indoprofen, Ketoprofen, Loxoprofen, Naproxen, Oxaprozin, Pranoprofen, And Suprofen. Examples of a suitable acetic acid derivative NSAID include, without limitation, Aceclofenac, Acemetacin, Actarit, Alcofenac, Aloxipirin, Amfenac, Aminophenazone, Antraphenine, Azapropazone, Benorilate, Benzydamine, Butibufen, Chlorthenoxacine, Choline Salicylate, Clometacin, Diclofenac, Emorfazone, Epirizole, Etodolac, Feclobuzone, Felbinac, Fenbufen, Fenclofenac, Glafenine, Indometacin, Ketorolac, Lactyl Phenetidin, Metamizole, Metiazinic Acid, Mofebutazone, Mofezolac, Nabumetone, Nifenazone, Niflumic Acid, Oxametacin, Pipebuzone, Propyphenazone, Proquazone, Protozininc Acid, Salicylamide, Sulindac, Tiaramide, Tinoridine, and Zomepirac. Examples of a suitable enolic acid (Oxicam) derivative NSAID include, without limitation, Droxicam, Isoxicam, Lornoxicam, Meloxicam, Piroxicam, and Tenoxicam. Examples of a suitable fenamic acid derivative NSAID include, without limitation, Flufenamic acid, Mefenamic acid, Meclofenamic acid, and Tolfenamic acid. Examples of a suitable selective COX-2 inhibitor include, without limitation, Celecoxib, Etoricoxib, Firocoxib, Lumiracoxib, Meloxicam, Parecoxib, Rofecoxib, and Valdecoxib.

The pharmaceutically active agents of the disclosure may be formulated into compositions as natural or salt forms. Pharmaceutically acceptable non-toxic salts include the base addition salts (formed with free carboxyl or other anionic groups), which may be derived from inorganic bases such as, for example, sodium, potassium, ammonium, calcium, or ferric hydroxides, and such organic bases as isopropylamine, trimethylamine, 2-ethylamino-ethanol, histidine, procaine, and the like. Such salts may also be formed as acid addition salts with any free cationic groups and will generally be formed with inorganic acids such as, for example, hydrochloric, sulfuric, or phosphoric acids, or organic acids such as acetic, citric, p-toluenesulfonic, methanesulfonic acid, oxalic, tartaric, mandelic, and the like. Salts of the disclosure include amine salts formed by the protonation of an amino group with inorganic acids such as hydrochloric acid, hydrobromic acid, hydroiodic acid, sulfuric acid, phosphoric acid, and the like. Salts of the disclosure may also include amine salts formed by the protonation of an amino group with suitable organic acids, such as p-toluenesulfonic acid, acetic acid, and the like.

In one aspect, the active pharmaceutical agent is a cannabinoid. As such, the disclosure provides compositions comprising at least one cannabinoid in an amount effective for treating a disease or disorder, and a pharmaceutically acceptable vehicle.

The bioavailability of cannabinoids, in particular CBD, ranges from 11 to 45% (mean 31%). Because bioavailability inversely correlates with the coefficient of variation of drug level concentrations in the patient, higher bioavailability provides tighter control of blood drug levels thus making dosing and resulting pharmacodynamics more predictable and less variable from patient to patient. In some embodiments, there are provided (non-aqueous) pharmaceutical solutions for nasal administration comprising: (a) a cannabinoid drug; (b) one or more natural or synthetic tocopherols or tocotrienols, or any combinations thereof, in an amount from about 30% to about 95% (w/w); (c) one or more alcohols or glycols, or any combinations thereof, in an amount from about 10% to about 70% (w/w); and (d) an alkyl glycoside, in a pharmaceutically-acceptable solution for administration to one or more nasal mucosal membranes of a patient. In some embodiments, the cannabinoid drug is dissolved in the one or more natural or synthetic tocopherols or tocotrienols, or any combinations thereof, in an amount from about 30% to about 95% (w/w); and the one or more alcohols or glycols, or any combinations thereof, in an amount from about 10% to about 70% (w/w). In some embodiments, the cannabinoid drug is selected from the group consisting of: THC (Δ9-tetrahydrocannabinol), CBD (Cannabidiol), CBG (Cannabigerol), CBC (Cannabichromene), CBGV (Cannabigerivarin), cannabinol (CBN), THCV (Tetrahydrocannabivarin), CBDV (Cannabidivarin), CBCV (Cannabichromevarin), CBGA (Cannabigerolic acid), THCA (Δ9-tetrahydrocannabinolic acid), CBDA (Cannabidiolic acid), CBCA (Cannabichromenenic acid), CBGVA (Cannabigerovarinic acid), HCVA (Tetrahydrocanabivarinic acid), CBDVA (Cannabidivarinic acid), and BCVA (Cannabichromevarinic acid), Cannabinol (CBN), any pharmaceutically-acceptable salts thereof, and any combinations thereof. In some embodiments, the cannabinoid drug is THC (Δ9-tetrahydrocannabinol) or CBD (Cannabidiol), or a pharmaceutically-acceptable salt thereof. In some embodiments, the solution contains about 1 to about 20% (w/v) of cannabinoid, e.g. about 1 to about 20% (w/v) of cannabidiol. In some embodiments, the formulation comprises equal concentrations of THC and CBD.

In some embodiments, the one or more natural or synthetic tocopherols or tocotrienols (collectively referred to as Vitamin E) are selected from the group consisting of: α-tocopherol, β-tocopherol, γ-tocopherol, δ-tocopherol, α-tocotrienol, β-tocotrienol, γ-tocotrienol, δ-tocotrienol, tocophersolan, Vitamin E TPGS, any isomers thereof, any esters thereof, any analogs or derivatives thereof, and any combinations thereof.

Alternatively, in some embodiments, Vitamin E may be replaced with food grade oils such as soybean oil, canola oil, olive oil, palm oil, coconut oil, corn oil, avocado oil, mustard oil, peanut oil, rice bran oil, safflower oil, sesame oil, sunflower oil, and Miglyol® 812 (neutral oil, triglycerides of medium chain fatty acids), polyethylene glycols, especially PEG 400-600 and any other solvent which doesn't degrade or solubilize the gelatin or potato starch shell, i.e., dimethyl isosorbide, surfactants, diethylene glycol monoethyl ether.

In some embodiments of the formulation, one or more alcohols may be included selected from the group consisting of: ethanol, propyl alcohol, butyl alcohol, pentanol, benzyl alcohol, any isomers thereof, or any combinations thereof. In some embodiments, the solution contains two or more alcohols, such as ethanol (1-25% (w/v)) and benzyl alcohol (1-25% (w/v)), or ethanol (10-22.5% (w/v)) and benzyl alcohol (7.5-12.5% (w/v)).

In some embodiments, the cannabinoid is present in the pharmaceutical composition in a concentration from about 1 mg/mL to about 200 mg/mL. In some embodiments, the one or more natural or synthetic tocopherols or tocotrienols, or any combinations thereof, is in an amount from about 30% to about 85% (w/w). In some embodiments, the one or more natural or synthetic tocopherols or tocotrienols, or any combinations thereof, is in an amount from about 50% to about 75% (w/w). In some embodiments, the one or more alcohols or glycols, or any combinations thereof, is in an amount from about 10% to about 55% (w/w), e.g. about 25% to about 40% (w/w). In some embodiments, the solution consists of cannabinoid (5-15% (w/v)), alkyl glycoside (0.01-1% (w/v)), vitamin E (45-65% (w/v)), ethanol (10-25% (w/v)) and benzyl alcohol (5-15% (w/v)). In some embodiments, the solution comprises at least about 0.01% (w/w) of an alkyl glycoside, e.g. about 0.01% to 3% (w/w) of an alkyl glycoside, such as dodecyl maltoside. In some embodiments, the solution consists of cannabinoid (5-35% (w/v)), dodecyl maltoside (0.01-5% (w/v)), vitamin E (45-65% (w/v)), ethanol (10-25% (w/v)) and benzyl alcohol (5-15% (w/v)); more particularly the solution may consist of cannabinoid (2-30% (w/v)), dodecyl maltoside (0.1-2.5% (w/v)), vitamin E (50-60% (w/v)), ethanol (15-22.5% (w/v)) and benzyl alcohol (7.5-12.5% (w/v)); and even more particularly, the solution may consist of a cannabinoid (20% (w/v)), dodecyl maltoside (0.15-0.5% (w/v)), vitamin E (50-60% (w/v)), ethanol (17-20% (w/v)) and benzyl alcohol (10-12% (w/v)).

Some embodiments described herein provide a method of treating a patient with a disorder which may be treatable with a cannabinoid drug, comprising: administering to one or more nasal mucosal membranes of a patient a pharmaceutical solution for nasal administration consisting of a cannabinoid drug, one or more natural or synthetic tocopherols or tocotrienols, or any combinations thereof, in an amount from about 30% to about 95% (w/w); one or more alcohols or glycols, or any combinations thereof, in an amount from about 10% to about 70% (w/w); and an alkyl glycoside. In some embodiments, the cannabinoid drug is dissolved in the one or more natural or synthetic tocopherols or tocotrienols, or any combinations thereof, in an amount from about 30% to about 95% (w/w); and the one or more alcohols or glycols, or any combinations thereof, in an amount from about 10% to about 70% (w/w). In some embodiments, the cannabinoid drug is selected from the group consisting of: THC (Δ9-tetrahydrocannabinol), CBD (Cannabidiol), CBG (Cannabigerol), CBC (Cannabichromene), CBGV (Cannabigerivarin), cannabinol (CBN), THCV (Tetrahydrocannabivarin), CBDV (Cannabidivarin), CBCV (Cannabichromevarin), CBGA (Cannabigerolic acid), THCA (Δ9-tetrahydrocannabinolic acid), CBDA (Cannabidiolic acid), CBCA (Cannabichromenenic acid), CBGVA (Cannabigerovarinic acid), HCVA (Tetrahydrocanabivarinic acid), CBDVA (Cannabidivarinic acid), and BCVA (Cannabichromevarinic acid), Cannabinol (CBN), any pharmaceutically-acceptable salts thereof, and any combinations thereof.

In some embodiments, the cannabinoid drug is CBD (Cannabidiol). In some embodiments, the solution contains about 1 to about 40% (w/v) of cannabinoid, e.g. about 1 to about 40% (w/v) of cannabidiol. In some embodiments, the one or more natural or synthetic tocopherols or tocotrienols are selected from the group consisting of: α-tocopherol, β-tocopherol, γ-tocopherol, δ-tocopherol, α-tocotrienol, β-tocotrienol, γ-tocotrienol, δ-tocotrienol, tocophersolan, any isomers thereof, any esters thereof, any analogs or derivatives thereof, and any combinations thereof. In some embodiments, the one or more alcohols are selected from the group consisting of: ethanol, propyl alcohol, butyl alcohol, pentanol, benzyl alcohol, any isomers thereof, or any combinations thereof. In some embodiments, the solution contains two or more alcohols, such as ethanol (1-25% (w/v)) and benzyl alcohol (1-25% (w/v)), or ethanol (10-22.5% (w/v)) and benzyl alcohol (7.5-12.5% (w/v)). In some embodiments, the cannabinoid is present in the pharmaceutical composition in a concentration from about 20 mg/mL to about 200 mg/mL. In some embodiments, the one or more natural or synthetic tocopherols or tocotrienols, or any combinations thereof, is in an amount from about 45% to about 85% (w/w). In some embodiments, the one or more natural or synthetic tocopherols or tocotrienols, or any combinations thereof, is in an amount from about 50% to about 75% (w/w). In some embodiments, the one or more alcohols or glycols, or any combinations thereof, is in an amount from about 15% to about 55% (w/w), e.g. about 25% to about 40% (w/w). In some embodiments, the solution consists of cannabinoid (5-15% (w/v)), alkyl glycoside (0.01-1% (w/v)), vitamin E (45-65% (w/v)), ethanol (10-25% (w/v)) and benzyl alcohol (5-15% (w/v)). In some embodiments, the solution comprises at least about 0.01% (w/w) of an alkyl glycoside, e.g. about 0.01% to 1% (w/w) of an alkyl glycoside, such as dodecyl maltoside. In some embodiments, the solution consists of THC (5-25% (w/v)), dodecyl maltoside (0.01-1% (w/v)), vitamin E (45-65% (w/v)), ethanol (10-25% (w/v)) and benzyl alcohol (5-15% (w/v)); more particularly the solution may consist of cannabinoid (9-11% (w/v)), dodecyl maltoside (0.1-0.5% (w/v)), vitamin E (50-60% (w/v)), ethanol (15-22.5% (w/v)) and benzyl alcohol (7.5-12.5% (w/v)); and even more particularly, the solution may consist of cannabinoid (10% (w/v)), dodecyl maltoside (0.15-0.3% (w/v)), vitamin E (50-60% (w/v)), ethanol (17-20% (w/v)) and benzyl alcohol (10-12% (w/v)). In some embodiments, the patient is human. In some embodiments, the cannabinoid is administered in a therapeutically effective amount from about 1 mg to about 40 mg.

In some embodiments, the cannabinoid is administered as in a dosage volume from about 10 μL to about 200 μL. In some embodiments, the administration of the pharmaceutical composition comprises spraying at least a portion of the therapeutically effective amount of the cannabinoid into at least one nostril. In some embodiments, the administration of the pharmaceutical composition comprises spraying at least a portion of the therapeutically effective amount of the cannabinoid into each nostril. In some embodiments, administration of the pharmaceutical composition comprises spraying a first quantity of the pharmaceutical composition into the first nostril, spraying a second quantity of the pharmaceutical composition into a second nostril, and optionally after a pre-selected time delay, spraying a third quantity of the pharmaceutical composition into the first nostril. In some embodiments, the method further comprises, optionally after a pre-selected time delay, administering at least a fourth quantity of the pharmaceutical composition to the second nostril. In some embodiments, nasal administration of the pharmaceutical composition begins at any time before or after onset of symptoms of a disorder which may be treatable with the pharmaceutical composition. In some embodiments, the treatment achieves bioavailability that is from about 50-125% (e.g. about 75-110%, or more particularly about 90-105%) of that achieved with the same cannabinoid administered orally.

In this context, it is intended that bioavailability be determined by a suitable pharmacodynamic method, such as comparison of area under the blood plasma concentration curve (AUC) for the nasally and orally administered drug. It is further understood that the percent bioavailability of the nasally administered cannabinoid may be determined by comparing the area under the blood plasma concentration curve obtained with one dose of the cannabinoid (e.g. 10 mg of nasal CBD) with another dose of the same cannabinoid administered orally (e.g. 10 mg of oral CBD), or an alternate dose taking into consideration the difference in dose. Thus, for the sake of illustration, a 10 mg nasal CBD dose that achieves an AUC that is precisely half of the AUC obtained with 5 mg of i.v. or orally administered CBD, would have a bioavailability of 50% relative to the mode of administration. In some embodiments, the disorder to be treated is neuropathic pain, for example from chemotherapy induced neuropathy or diabetic neuropathy. In some embodiments, the solution and treatment with the solution are substantially non-irritating and well-tolerated.

In some embodiments, the pharmaceutical composition for nasal administration comprises: a cannabinoid drug; one or more natural or synthetic tocopherols or tocotrienols, or any combinations thereof, in an amount from about 30% to about 95% (w/w); and one or more alcohols or glycols, or any combinations thereof, in an amount from about 5% to about 70% (w/w), preferably about 10% to about 70% (w/w) in a pharmaceutically-acceptable formulation for administration to one or more nasal mucosal membranes of the patient. In some embodiments the cannabinoid drug is dissolved in the one or more natural or synthetic tocopherols or tocotrienols, or any combinations thereof, in an amount from about 30% to about 95% (w/w); and the one or more alcohols or glycols, or any combinations thereof, in an amount from about 5% to about 70% (w/w), preferably about 10% to about 70% (w/w). In some embodiments, the cannabinoid drug is dissolved in a carrier system. In some embodiments, at least part of the cannabinoid drug is in a form comprising cannabinoid microparticles, nanoparticles or combinations thereof. In some embodiments, the composition is substantially free of cannabinoid microparticles, nanoparticles or combinations thereof.

In some embodiments, the cannabinoid drug is selected from the group consisting of: THC (Δ9-tetrahydrocannabinol), CBD (Cannabidiol), CBG (Cannabigerol), CBC (Cannabichromene), CBGV (Cannabigerivarin), cannabinol (CBN), THCV (Tetrahydrocannabivarin), CBDV (Cannabidivarin), CBCV (Cannabichromevarin), CBGA (Cannabigerolic acid), THCA (Δ9-tetrahydrocannabinolic acid), CBDA (Cannabidiolic acid), CBCA (Cannabichromenenic acid), CBGVA (Cannabigerovarinic acid), HCVA (Tetrahydrocanabivarinic acid), CBDVA (Cannabidivarinic acid), and BCVA (Cannabichromevarinic acid), Cannabinol (CBN), any pharmaceutically-acceptable salts thereof, and any combinations thereof. In some embodiments, the cannabinoid drug is CBD (Cannabidiol), or any pharmaceutically-acceptable salts thereof. In some embodiments, the cannabinoid drug is cannabinoid, or a pharmaceutically-acceptable salt thereof. In some embodiments, the cannabinoid drug comprises cannabinoid microparticles, nanoparticles, or combinations thereof. In some embodiments, the cannabinoid nanoparticles have an effective average particle size of less than about 5000 nm. In some embodiments, the cannabinoid drug is substantially free of cannabinoid microparticles, nanoparticles or combinations thereof.

In some embodiments, the one or more natural or synthetic tocopherols or tocotrienols are selected from the group consisting of: α-tocopherol, β-tocopherol, γ-tocopherol, δ-tocopherol, α-tocotrienol, β-tocotrienol, γ-tocotrienol, δ-tocotrienol, tocophersolan, any isomers thereof, any esters thereof, any analogs or derivatives thereof, and any combinations thereof. In some embodiments, a synthetic tocopherol can include Vitamin E TPGS (Vitamin E polyethylene glycol succinate). In some embodiments, on the other hand, synthetic tocopherols exclude tocopherols covalently bonded or linked (e.g. through a diacid linking group) to a glycol polymer, such as polyethylene glycol). Thus, in some embodiments, the compositions described herein exclude Vitamin E TPGS.

In some embodiments, one or more alcohols are selected from the group consisting of: ethanol, propyl alcohol, butyl alcohol, pentanol, benzyl alcohol, any isomers thereof, or any combinations thereof. In some embodiments, the one or more glycols are selected from the group consisting of: ethylene glycol, propylene glycol, butylene glycol, pentylene glycol, any isomers thereof, and any combinations thereof. In some preferred embodiments, the glycols exclude glycol polymers. In some preferred embodiments, the glycols exclude glycol polymers having an average molecular weight of greater than 200. In some embodiments, the glycols exclude polyethylene glycol having an average molecular weight of greater than about 200.

In some embodiments, the cannabinoid drug is present in the carrier system in a concentration from about 1 mg/mL to about 600 mg/mL. In some embodiments, the cannabinoid drug is present in a carrier system in a concentration from about 10 mg/mL to about 250 mg/mL. In some embodiments, the cannabinoid is present in a carrier system in a concentration from about 20 mg/mL to about 50 mg/mL. As with most drug substances, individuals with more body mass will require more cannabinoid experience its effects. By way of example only, a rule of thumb to determine the proper CBD dosage might be to take 1-6 mg of CBD for every 10 pounds of body weight based on the individual's level of pain. For example, 20 mg-33 mg might be a starting dosage for a 200 lb patient, while 15 mg-25 mg might be appropriate for another who weighs 150 lb.

In some embodiments, the carrier system comprises one or more natural or synthetic tocopherols or tocotrienols, or any combinations thereof, in an amount from about 45% to about 85% (w/w). In some embodiments, the carrier system comprises one or more natural or synthetic tocopherols or tocotrienols, or any combinations thereof, in an amount from about 60% to about 75% (w/w). In some embodiments, the carrier system comprises one or more natural or synthetic tocopherols or tocotrienols, or any combinations thereof, in an amount of about 70% (w/w).

In some embodiments, the carrier system comprises one or more alcohols or glycols, or any combinations thereof, in an amount from about 10% to about 70% (w/w). In some embodiments, the carrier system comprises one or more alcohols or glycols, or any combinations thereof, in an amount from about 15% to about 55% (w/w). In some embodiments, the carrier system comprises one or more alcohols or glycols, or any combinations thereof, in an amount from about 25% to about 40% (w/w). In some embodiments, the carrier system comprises one or more alcohols or glycols, or any combinations thereof, in an amount of about 30% (w/w).

In some embodiments, the composition comprises at least one additional ingredient selected from the group consisting of: active pharmaceutical ingredients; enhancers; excipients; and agents used to adjust the pH, buffer the composition, prevent degradation, and improve appearance, odor, or taste.

In some embodiments, the composition comprises one or more additional excipients, such as one or more parabens, one or more povidones, one or more poloxamers, sodium alginate, pectin, and/or one or more alkyl glycosides.

The invention also discloses a method of treating a patient (i.e., mammal, such as a human, veterinary or companion animal, such as a cat or dog) with a disorder that may be treatable with a cannabinoid drug.

The term “in need of treatment” and the term “in need thereof” when referring to treatment are used interchangeably and refer to a subject that will benefit from treatment.

The compositions of the disclosure may be used to treat a disease or disorder, or ameliorate symptoms of a disease or disorder, including pain (chronic and neuropathic), diabetes, cancer, neurodegenerative diseases, epilepsy, insomnia, anxiety and/or social anxiety disorder, migraine, fibromyalgia, irritable bowel syndrome, and related conditions represent clinical endocannabinoid (eCB) deficiency syndromes (CEDS), depression, schizophrenia, multiple sclerosis (MS), Huntington's disease, Parkinson's disease, anorexia, and chronic motion sickness.

In some embodiments, the patient is a human. In some embodiments, the method comprises: administering to one or more nasal mucosal membranes of a patient a pharmaceutical composition for nasal administration comprising a cannabinoid drug; one or more natural or synthetic tocopherols or tocotrienols, or any combinations thereof, in an amount from about 30% to about 95% (w/w); and one or more alcohols or glycols, or any combinations thereof, in an amount from about 5% to about 70%, preferably about 10% to about 70% (w/w). In some embodiments, the cannabinoid is dissolved in the one or more natural or synthetic tocopherols or tocotrienols, or any combinations thereof, in an amount from about 30% to about 95% (w/w); and the one or more alcohols or glycols, or any combinations thereof, in an amount from about 5% to about 70%, preferably about 10% to about 70% (w/w). In some embodiments, the cannabinoid drug is dissolved in a carrier system. In some embodiments, the cannabinoid drug includes cannabinoid microparticles, nanoparticles, or combinations thereof. In some embodiments, the composition is substantially free of cannabinoid microparticles, nanoparticles or combinations thereof.

In some embodiments, the cannabinoid drug is selected from the group consisting of: THC (Δ9-tetrahydrocannabinol), CBD (Cannabidiol), CBG (Cannabigerol), CBC (Cannabichromene), CBGV (Cannabigerivarin), cannabinol (CBN), THCV (Tetrahydrocannabivarin), CBDV (Cannabidivarin), CBCV (Cannabichromevarin), CBGA (Cannabigerolic acid), THCA (Δ9-tetrahydrocannabinolic acid), CBDA (Cannabidiolic acid), CBCA (Cannabichromenenic acid), CBGVA (Cannabigerovarinic acid), HCVA (Tetrahydrocanabivarinic acid), CBDVA (Cannabidivarinic acid), and BCVA (Cannabichromevarinic acid), Cannabinol (CBN), any pharmaceutically-acceptable salts thereof, and any combinations thereof. In some embodiments, the cannabinoid drug is CBD (Cannabidiol). In some embodiments, the cannabinoid drug is fully dissolved in a single phase comprising one or more one or more natural or synthetic tocopherols or tocotrienols and one or more alcohols or glycols. In some embodiments, the cannabinoid drug comprises cannabinoid microparticles, nanoparticles, or combinations thereof. In some such embodiments, the composition further comprises water. In some embodiments, the cannabinoid nanoparticles have an effective average particle size of less than about 5000 nm. In some embodiments, the composition is substantially free of cannabinoid microparticles, nanoparticles or combinations thereof.

In some embodiments, the one or more natural or synthetic tocopherols or tocotrienols are selected from the group consisting of: α-tocopherol, β-tocopherol, γ-tocopherol, δ-tocopherol, α-tocotrienol, β-tocotrienol, γ-tocotrienol, δ-tocotrienol, tocophersolan, any isomers thereof, any esters thereof, any analogs or derivatives thereof, and any combinations thereof.

In some embodiments, the one or more alcohols are selected from the group consisting of: ethanol, propyl alcohol, butyl alcohol, pentanol, benzyl alcohol, any isomers thereof, and any combinations thereof. In some embodiments, the one or more glycols are selected from the group consisting of: ethylene glycol, propylene glycol, butylene glycol, pentylene glycol, any isomers thereof, and any combinations thereof. In some embodiments, the alcohol or glycol is free of water (dehydrated, USP). In some embodiments, the alcohol is ethanol (dehydrated, USP).

In some embodiments, the cannabinoid drug is present in the carrier system in a concentration from about 1 mg/mL to about 600 mg/mL. In some embodiments, the cannabinoid drug is present in the carrier system in a concentration of from about 10 mg/mL to about 250 mg/mL. In some embodiments, the cannabinoid drug is present in the carrier system in a concentration of from about 20 mg/mL to about 50 mg/mL.

In some embodiments, the carrier system comprises one or more natural or synthetic tocopherols or tocotrienols, or any combinations thereof, in an amount from about 45% to about 85% (w/w). In some embodiments, the carrier system comprises one or more natural or synthetic tocopherols or tocotrienols, or any combinations thereof, in an amount from about 60% to about 75% (w/w). In some embodiments, the carrier system comprises one or more natural or synthetic tocopherols or tocotrienols, or any combinations thereof, in an amount of about 70% (w/w).

In some embodiments, the carrier system comprises one or more alcohols or glycols, or any combinations thereof, in an amount from about 15% to about 55% (w/w). In some embodiments, the carrier system comprises one or more alcohols or glycols, or any combinations thereof, in an amount from about 25% to about 40% (w/w). In some embodiments, the carrier system comprises one or more alcohols or glycols, or any combinations thereof, in an amount from about 30% (w/w).

In some embodiments, the composition comprises at least one additional ingredient selected from the group consisting of: active pharmaceutical ingredients; enhancers; excipients; and agents used to adjust the pH, buffer the composition, prevent degradation, and improve appearance, odor, or taste.

In some embodiments, the composition is in a pharmaceutically-acceptable spray formulation, and further comprising administering the composition to one or more nasal mucosal membranes of the patient. In some embodiments, the therapeutically effective amount is from about 1 mg to about 20 mg of the cannabinoid. In some embodiments, the pharmaceutical composition is in a pharmaceutically-acceptable spray formulation having volume from about 100 μL to 200 μL.

In some embodiments, the administration of the composition comprises spraying at least a portion of the therapeutically effective amount of the composition into at least one nostril. In some embodiments, the administration of the composition comprises spraying at least a portion of the therapeutically effective amount of the composition into each nostril. In some embodiments, the administration of the composition comprises spraying a first quantity of the composition into the first nostril, spraying a second quantity of the composition into a second nostril, and optionally after a pre-selected time delay, spraying a third quantity of the composition into the first nostril. Some embodiments further comprise, optionally after a pre-selected time delay, administering at least a fourth quantity of the composition to the second nostril.

In some embodiments, the administration of the composition begins at any time before or after onset of symptoms of a disorder which may be treatable with the composition.

The oral bioavailability of cannabinoids, particularly CBD, ranges from approximately 13 to 19%. Administering an oil formulation directly into the oral cavity has not been a practical means of administering oil soluble drugs. Since the oil doesn't mix with saliva, the oil tends to remain in the mouth providing an uncomfortable physical sensation. Soft gel capsules provide an effective means of oral delivery for oil-soluble (i.e., hydrophobic) drugs. Oil soluble drugs can be dissolved in a pharmaceutically acceptable oil formulation and encapsulated in the soft gel capsules. Soft gel capsules typically consist of a single piece shell consisting of gelatin, water, an opacifier and a plasticizer such as glycerin. An alternative to gelatin based capsules are potato starch matrix capsules. Potato starch matrix is a smooth, transparent substance resembling gelatin, which is neutral in taste and color, easily digestible and of plant origin, and therefore, the concerns of certain bovine related diseases are not an issue, and they offer a gelatin free alternative for vegetarians and vegans, commercially available from Swiss Caps AG under the name Vegagels®. Soft gel capsules can be enteric-coated to alter the location within the GI tract at which the contents of the gel are released.

The formation and filling of soft gelatin capsules is accomplished using a rotary die process which comprises continuously feeding two ribbons of gelatin into a rotating die assembly where the two halves of a capsule are simultaneously formed. The ribbons converge adjacent to a fill injector which measures and dispenses the appropriate volume of fill material into the capsules. The filled capsules are subsequently sealed as the die assembly rotates. This process permits accurate and reproducible fill uniformity. A number of companies offer filled gel cap manufacturing services on a contract basis.

Ideally, the bioavailability of poorly absorbed drugs can be up improved by addition of an absorption enhancer. The principal problem and main reason for failure of absorption enhancers to increase oral bioavailability of drugs results from the fact that once the mixture of drug and absorption enhancer enters the GI tract, the absorption enhancer and drug diffuse throughout the contents of the GI tract upon dissolution of the gel cap. Since the performance of the absorption enhancer is concentration dependent, dilution of the absorption enhancer as it dissolves into the aqueous GI tract contents is quickly diminished, thus dropping below the optimized effective ratio or concentration of absorption enhancer to drug.

In the present invention, it was discovered that certain hydrophobic absorption enhancers, in particular alkylsaccharides soluble in oil formulations may be added to the formulation of oil soluble drug to be encapsulated in the soft gel, and following administration of the soft gel capsules to the GI tract and subsequent solubilization of the gelatin capsule within the GI tract, the absorption enhancer remains in proximity to drug dissolved within the oil globules formed upon breakup of the soft gel capsules at substantially the same concentrations and ratio present in the original oil formulation. Thus the absorption enhancer is not diluted to a concentration below its effective concentration in the GI tract. When the oil globules come in contact with the mucosal surface of the GI tract the hydrophobic drug and hydrophobic absorption enhancer element is delivered at the mucosal surface at the effective or near optimal concentrations and ratio of absorption enhancer to drug. Thus, the present invention provides a means of increasing the oral bioavailability of oil-soluble (hydrophobic) drugs such as cannabinoids by maintaining the absorption enhancer and drug in close physical proximity until the oil droplets present in the aqueous G.I. contents come in contact with the mucosal surface of the GI tract. Because cannabinoids are very hydrophobic, they are ideal candidates for oral drug delivery according to the present invention.

The present invention employs the use of alkylsaccharides surfactants which have substantial hydrophobic character such as to be soluble in pharmaceutically acceptable oils. These include, but are not limited to dodecyl maltoside, octyl glucoside, dodecyl maltoside, tetradecyl maltoside, hexadecimal maltoside, octadecyl maltoside, decyl maltoside, sucrose dodecanoate, sucrose tetradecanoate, sucrose octadecanoate and mixed sucrose mono and dioctadecanoate. Alkylsaccharide concentrations useful in the present invention range from 0.01% to 20%, 0.05% to 10%, 0.1% to 5%.

The present invention employs the use of an organic solvent that is immiscible with the aqueous contents of the GI tract. Thus in one embodiment of the present invention, a cannabinoid and an oil soluble alkylsaccharide are dissolved in a pharmaceutically acceptable organic solvent such as a food grade oil, including, but not limited to, soybean oil, canola oil, olive oil, palm oil, coconut oil, corn oil, avocado oil, mustard oil, peanut oil, rice bran oil, safflower oil, sesame oil, sunflower oil, and Miglyol® 812 (neutral oil, triglycerides of medium chain fatty acids, polyethylene glycols, especially PEG 400-600, and any other solvent which doesn't degrade or solubilize the gelatin or potato starch shell, i.e., dimethyl isosorbide, surfactants, diethylene glycol monoethyl ether and poloxamers. Poloxamers are non-ionic poly (ethylene oxide) (PEO)-poly (propylene oxide) (PPO) copolymers. They are used in pharmaceutical formulations as surfactants, emulsifying agents, solubilizing agent, dispersing agents, and in vivo absorbance enhancer. Examples include poloxamers 407, 124, and 188.

The present invention employs the use of alkylsaccharides surfactants which have substantial hydrophobic character such as to be soluble in pharmaceutically acceptable oils. These include, but are not limited to alkylsaccharides. Alkylsaccharide concentrations useful in the present invention range from 0.01% to 20%, 0.05% to 10%, 0.1% to 5%.

Cannabinoids suitable for delivery according to the present invention include (THC (Δ9-tetrahydrocannabinol), CBD (Cannabidiol), CBG (Cannabigerol), CBC (Cannabichromene), CBGV (Cannabigerivarin), cannabinol (CBN), THCV (Tetrahydrocannabivarin), CBDV (Cannabidivarin), CBCV (Cannabichromevarin), CBGA (Cannabigerolic acid), THCA (Δ9-tetrahydrocannabinolic acid), CBDA (Cannabidiolic acid), BCA (Cannabichromenenic acid), CBGVA (Cannabigerovarinic acid), HCVA (Tetrahydrocanabivarinic acid), CBDVA (Cannabidivarinic acid), and BCVA (Cannabichromevarinic acid), Cannabinol (CBN), any pharmaceutically-acceptable salts thereof, and any combinations thereof.

The oral bioavailability of CBD is 13 to 19%. Soft Capsules provide an effective means of oral delivery for poorly water-soluble drugs because they can be filled with liquid ingredients that increase solubility that will be difficult to include solid tablet. Soft gel capsules typically consist of a single piece shell consisting of gelatin, water, an opacifier and a plasticizer such as glycerin. An alternative to gelatin base capsules are potato starch matrix capsules. Potato starch matrix is a smooth, transparent substance resembling gelatin, which is neutral in taste and color, easily digestible and of plant origin, and therefore, the concerns of certain bovine related diseases are not an issue, and they offer a gelatin free alternative for vegetarians and vegans, currently available from Swiss Caps AG under the name Vegagels®. Soft gel capsules can be enteric-coated to alter the location within the GI tract at which the contents of the gel are released.

The formation and filling of soft gelatin capsules is accomplished using a rotary die process which comprises continuously feeding two ribbons of gelatin into a rotating die assembly where the two halves of a capsule are simultaneously formed. The ribbons converge adjacent to a fill injector which measures and dispenses the appropriate volume of fill material into the capsules. The filled capsules are subsequently sealed as the die assembly rotates. This process permits accurate and reproducible fill uniformity. A number of companies offer filled gel cap manufacturing services on a contract basis.

The principal problem and main reason for failure of absorption enhancers to increase oral bioavailability of drugs results from the fact that once the mixture of drug and absorption enhancer enters the GI tract, the absorption enhancer and drug diffuse throughout the contents of the GI tract upon dissolution of the gel. Since the performance of the absorption enhancer is concentration dependent, dilution of the absorption enhancer as it dissolves into the aqueous GI tract contents is quickly diminished, thus dropping below the optimized effective ratio of absorption enhancer to drug.

In the present invention, it was discovered that if the drug of interest is hydrophobic and if the absorption enhancer is sufficiently hydrophobic to dissolve well into oil-based formulations, when the mixture of surfactant drug present in the oil solution is released into the GI tract upon dissolution of the gel cap, the drug and absorption enhancer remain dissolved within the oil globules formed upon breakup of the soft gel capsules at substantially the same concentrations and ratio present in the original oil formulation. When the oil globules come in contact with the mucosal surface of the GI tract the hydrophobic drug and hydrophobic absorption enhancer element delivered at the surface at the mucosal surface at the proper or near optimal concentrations and ratio of absorption enhancer to drug. Because cannabinoids are very hydrophobic, they are ideal candidates for oral drug delivery according to the present invention.

Additional embodiments, uses, and advantages of the invention will become apparent to the person skilled in the art upon consideration of the disclosure set forth herein.

The following example is provided to further illustrate the advantages and features of the present invention, but it is not intended to limit the scope of the invention. While this example is typical of those that might be used, other procedures, methodologies, or techniques known to those skilled in the art may alternatively be used.

### EXAMPLES

### Example 1

### Preparation of Oil-Based Cannabinoid Formulations

Preparation of Materials

Oil-based cannabinoid formulations comprising vitamin E (optionally substituted with another oil as described herein), and ethyl alcohol (optionally substituted with another alcohol as described herein), and less than about 1% water, as illustrated in Example 1, are prepared to which is added one or more alkylsaccharides such as dodecyl maltoside, tetradecyl maltoside, and/or sucrose dodecanoate in an amount of 0.1% to 5%. The resulting formulation may be inserted into a soft gel cap.

TABLE 1
Examples of Oil-Based Cannabinoid Formulations

| Cannabinoid | Vehicle Formulation |
|---|---|
| Cannabidiol (CBD) | Vitamin E (75%), Ethanol (15%), |
| | propylene glycol (10%) |
| Tetrahydrocannabinol (THC) | Vitamin E (70%), Ethanol (15%), |
| | propylene glycol (15%) |
| Cannabidiol (CBD) + | Vitamin E (80%), Ethanol (15%), |
| Tetrahydrocannabinol (THC) | propylene glycol (5%) |
| 1:1 mixture 9 | |
| Tetrahydrocannabivarin THCV | Vitamin E (75%), Ethanol (15%), |
| | propylene glycol (10%) |
| Cannabidivarin CBDV | Vitamin E (75%), Ethanol (15%), |
| | propylene glycol (10%) |
| Cannabigerol CBG | Vitamin E (75%), Ethanol (15%), |
| | propylene glycol (10%) |
| Cannabichromene CBC | Vitamin E (70%), Ethanol (15%), |
| | propylene glycol (15%) |
| Cannabinol CBN | Vitamin E (70%), Ethanol (15%), |
| | propylene glycol (15%) |
| Tetrahydrocannabinolic | Vitamin E (80%), Ethanol (15%), |
| Acid THCA | propylene glycol (5%) |
| Cannabigerolic | Vitamin E (80%), Ethanol (15%), |
| Acid CBGa | propylene glycol (5%) |

### Example 2

### Preparation of Aqueous Cannabinoid Suspension Formulation

In general, cannabinoid suspensions are formulated by combining a cannabinoid with a pharmaceutically acceptable carrier oil formulation. The carrier oil formulation is prepared by combining pharmaceutically acceptable oil with one or more lower alcohols or glycols with water, adding a natural or synthetic tocopherol or tocotrienol, heating the mixture until the tocopherol or tocotrienol is dissolved, adding one or more parabens and mixing until the parabens are dissolved and cooling the carrier. Once the cannabinoid is added to the carrier, additional excipients, such as surfactants, can optionally be added and dissolved in the carrier. The suspension is then brought up to its final mass volume with water.

By way of example, cannabidiol suspension is formulated by the foregoing general process in a carrier that is generated by combining propylene glycol USP and purified water USP, then adding vitamin E polyethylene glycols succinate NF, then mixing and heating the combined ingredients to about 45° C. as summarized in Table 2 below. Mixing is continued until the vitamin E polyethylene glycol succinate is fully dissolved. The carrier is then cooled to 25-25° C. The two concentrations of cannabinoid are separately added to the two batches of formulation. Polyvinylpyrrolidone povidone USP/NF is then added to the mixture and mixed until fully dissolved. The suspension is then brought up to weight with purified water USP. The suspension is then mixed until homogeneous, sampled for in process testing, and packaged in three mL amber glass bottles. The appearance of the suspension is that of an opaque white liquid. The suspension is subjected to stability testing at 25° C./60% relative humidity for three months in the appearance is observed and recorded monthly.

TABLE 2
Cannabidiol Suspension Composition

| Component | Function | Amount (mg) |
|---|---|---|
| Cannabidiol | Active | 100.0 |
| Methyl paraben | Preservative | 2.0 |
| Propyl paraben | Preservative | 0.5 |
| Dodecyl maltoside | Absorption enhancer | 2.5 |
| Vitamin E TPGS | Dispersant | 10.0 |
| Propylene glycol | Dispersant | 100.0 |
| Povidone | Suspending agent | 5.0 |
| Water | Carrier | q.s. to 1.0 mL |

### Example 3

### Oral Buccal/Palatal and Dermal Compositions

Pieces of palatal or buccal tissue are defrosted in isotonic saline solution and clamped into position between the donor and the receptor compartment of a static vertical diffusion cell, based on the Franz design (exposed area_0.78 cm2). The receptor medium, consisting of 2.2 ml of isotonic saline solution, is maintained at 37° C. by a circulating water pump, and constantly stirred with a teflon-coated magnetic bar. The donor side of the tissue is hydrated for 30 min with the receptor medium and subsequently dried with a cotton swab. The donor is then filled with 1 ml of the solution under test. The test solutions contain CBD 10% (w/v) in different vehicles (Table 2). Samples of 100 ml are withdrawn each hour from the receptor compartment over a period of 8 h (replacing them with an equivalent volume of fresh solution). The results are corrected for dilution effect. The steady-state flux of CBD is calculated from the slope of the linear portion of plots of cumulative amount penetrated versus time. At the end of the experiments, CBD is extracted overnight at room temperature by soaking the tissues in 2 ml of 20% (w/v) sodium chloride solution, and subsequently in 2 ml of distilled water. The extraction suspensions are centrifuged and the supernatant filtered through a membrane filter (0.45 mm, polypropylene micro-centrifuge tube filters, Whatman, Maidstone, England). All the samples are analyzed by high performance thin-layer chromatography (HPTLC) for CBD content. Experiments are generally performed in hextuplicate.

High performance thin-layer chromatography procedure is carried out as follows. First, 2 ml of the samples (aqueous receptors, CBD extracted from the tissue and unpenetrated donors at the end of the experiment), is spotted on HPTLC plates (silica gel 60, 10_20 cm without concentration zone), obtained from Merck (Darmstadt, Germany), using a Linomat IV (CAMAG, Muttenz, Switzerland). Separations are carried out in developing chambers (CAMAG), presaturated with the developing solvent system, consisting of butanol:acetone:glacial acetic acid:water (7:5:2:1 v:v). The solvent front is allowed to migrate to 4.0 cm above the origin. After drying the plates at 60° C. on a hotplate (CAMAG) for 40 min, they are scanned with a CAMAG TLC Scanner II at 225 nm, in the Refl-Abs mode, and the signals are integrated with a CAMAG SP4290 integrator. Standard solutions are applied on each plate for mass calibration purposes.

TABLE 3
Flux of CBD Across Buccal and Palatal
Mucosa With Different Alkylsaccharides

| | Sample Proforma Flux Ratios | |
|---|---|---|
| | Vehicle (mg/cm2/hour)/Vitamin E | |
| Sample Proforma Formulations | (mg/cm2/hour) | |
| Vehicle | Buccal | Palatal |
| Transcutol ®:water (50:50) | 11 | 5 |
| Ethanol:water (50:50) | 3 | 1 |
| Dodecylmaltoside (1.5% w/v) | 20 | 10 |
| Vit. E:Ethanol (75:25) | | |
| L-1695 (1.5% w/v) | 22 | 15 |
| Vit. E:Ethanol (75:25) | | |
| P-1670d (1.5% w/v) | 1 | 1 |
| Vit. E:Ethanol (75:25) | | |
| S-1670d (1.5% w/v) | 1 | 1 |
| Vit. E:Ethanol (75:25) | | |
| O-1570 (1.5% w/v) | 1 | 1 |
| Vit. E:Ethanol (75:25) | | |
| Tetradecylmaltoside | 20 | 10 |
| (1.5% w/v) Vit. E:Ethanol | | |
| (75:25) | | |
| Vit. E:Ethanol | 1 | 1 |
| (75:25) (control) | | |

TABLE 4
Physical Chemical Characteristics
of Sucrose Fatty Acid Esters (SE)

| | | | Ester composition (%) | |
|---|---|---|---|---|
| | | | | Di-, tri-, |
| SE | Fatty acid (carbon skeleton) | HLB | Mono ester | polyester |
| S-1670 | Stearic acid and parenthesis | 16 | 75 | 25 |
| | (18:0) | | | |
| O-1570 | Oleic acid (18:1) | 15 | 70 | 30 |
| P-1670 | Palmitic acid (16:0) | 16 | 80 | 20 |
| L-1695 | Lauric acid (12:0) | 16 | 80 | 20 |

Mitsubishi-Kasai Food Corporation 1987

### Example 4

### Soft Gel Cap Cannabidiol Formulations for Oral Administration

An oil formulation of cannabidiol containing 20% cannabidiol, 10% ethanol, 10% benzyl alcohol, 1% dodecyl maltoside and 59% vitamin E is prepared. The contents of a commercially available soft gel fish oil dietary supplement is removed using a fine gauge hypodermic syringe. The soft gel capsule is flushed with anhydrous ethanol and the cannabinol oil formulation is inserted into the empty soft gel capsule carefully removing any air bubbles using the syringe as necessary. The puncture hole caused by the hypodermic syringe is sealed by putting a small drop of water, approximately 10 μL, over the puncture and orienting the gel in such a way as to allow the water drop to remain in contact with the puncture site for at least three minutes to slightly solubilize the gel surface in the vicinity of the puncture thus sealing it from leakage. This manual approach to preparing soft gel caps containing cannabinol provides prototype gel caps to simulate materials produced by the automated production of soft gel caps provided by contract research manufacturers.

Although the invention has been described with reference to the above examples, it will be understood that modifications and variations are encompassed within the spirit and scope of the invention. Accordingly, the invention is limited only by the following claims.

1. A composition comprising an oil-soluble hydrophobic drug, an oil-soluble hydrophobic surfactant, and a pharmaceutically acceptable carrier, wherein the drug and the surfactant are dissolved in the pharmaceutically acceptable carrier.

2. The composition of claim 1, wherein the drug is a cannabinoid, an antiviral agent or a calcitonin gene-related peptide (CGRP) antagonist.

3. The composition of claim 1, wherein the pharmaceutically acceptable carrier is an oil.

4. The composition of claim 1, wherein the composition is substantially anhydrous.

5. The composition of claim 4, wherein the composition comprises less than about 5, 4, 3, 2, 1, 0.5, or 0.1% water.

6. The composition of claim 1, wherein the composition is formulated for nasal, topical, sublingual or oral delivery.

7. The composition of claim 6, wherein the composition is formulated as a liquid, cream, balm, ointment or capsule.

8. The composition of claim 7, wherein the capsule is a soft gel capsule.

9. The composition of claim 2, wherein the cannabinoid is selected from the group consisting of (THC (Δ9-tetrahydrocannabinol), CBD (Cannabidiol), CBG (Cannabigerol), CBC (Cannabichromene), CBGV (Cannabigerivarin), cannabinol (CBN), THCV (Tetrahydrocannabivarin), CBDV (Cannabidivarin), CBCV (Cannabichromevarin), CBGA (Cannabigerolic acid), THCA (Δ9-tetrahydrocannabinolic acid), CBDA (Cannabidiolic acid), CBCA (Cannabichromenenic acid), CBGVA (Cannabigerovarinic acid), CVA (Tetrahydrocanabivarinic acid), CBDVA (Cannabidivarinic acid), and BCVA (Cannabichromevarinic acid), Cannabinol (CBN), any pharmaceutically-acceptable salts thereof, and any combination thereof.

10. The composition of claim 3, wherein the oil is selected from the group consisting of Vitamin E (i.e., α-tocopherol, β-tocopherol, γ-tocopherol, δ-tocopherol, α-tocotrienol, β-tocotrienol, γ-tocotrienol, δ-tocotrienol, tocophersolan, any isomers thereof, any esters thereof, any analogs or derivatives thereof, and any combinations thereof), soybean oil, canola oil, olive oil, palm oil, coconut oil, corn oil, avocado oil, mustard oil, peanut oil, rice bran oil, safflower oil, sesame oil, sunflower oil, and any combination thereof.

11. The composition of claim 1, wherein the hydrophobic surfactant is selected from the group consisting of dodecyl maltoside, octyl glucoside, tridecyl maltoside, tetradecyl maltoside, hexadecimal maltoside, octadecyl maltoside, decyl maltoside, sucrose dodecanoate, sucrose tetradecanoate, sucrose octadecanoate and mixed sucrose mono and dioctadecanoate, and octyl glucoside.

12. The composition of claim 1, wherein the pharmaceutically acceptable carrier is selected from the group consisting of Miglyol® 812, triglycerides of medium chain fatty acids, polyethylene glycols including PEG 400-600 or poloxamers, and any combination thereof.

13. The composition of claim 1, wherein the composition further comprises an alcohol selected from the group consisting of ethanol, propyl alcohol, butyl alcohol, pentanol, benzyl alcohol, any isomers thereof, and any combination thereof.

14. The composition of claim 1, wherein the surfactant is an alkylsaccharide having an alkyl chain length of from about 10 to 18 carbons joined by glycoside bond to a sugar moiety.

15. The composition of claim 14, wherein the alkylsaccharide is an α-D-maltoside, β-D-maltoside, or combination thereof.

16. A soft gel capsule comprising the composition of claim 1.

17. An aqueous emulsion composition comprising water, a cannabinoid and an alkylsaccharide.

18. The composition of claim 17, wherein the composition if formulated for topical administration.

19. The composition of claim 18, wherein the alkylsaccharide is present in a concentration of about 0.05% to 2.5%.

20. The composition of claim 19, wherein the alkylsaccharide is selected from the group consisting of n-Dodecyl-maltoside, n-tetradecyl-maltoside, and sucrose octadecanoate.

21. The composition of claim 17, wherein the emulsion comprises about 56.47% (w/v) vitamin E, about 10.5% (w/v) benzyl alcohol, about 20% (w/v) cannabinoid, about 0.25% (w/v) dodecyl maltoside, and optionally dehydrated ethanol.

22. A method of treating a subject with a disorder which is treatable with a cannabinoid drug, comprising administering to the subject a composition of claim 1.

23-57. (canceled)

58. The composition of claim 2, wherein the antiviral compound a nucleoside analog.

59. The composition of claim 58, wherein the nucleoside analog is selected from the group consisting of remdesivir, abacavir, acyclovir, adefovir, brivudine, cidofovir, clevudine, didanosine, edoxudine, emtricitabine, entecavir, famciclovir, floxuridine, ganciclovir, idoxuridine, inosine pranobex, lamivudine, penciclovir, sorivudine, stavudine, ribavirin, telbivudine, tenofovir, trifluridine, valacyclovir, valganciclovir, vidarabine, zalcitabine, and zidovudine.

60. The composition of claim 59, wherein the nucleoside analog is remdesivir.

61. The composition of claim 2, wherein the CGRP antagonist is selected from the group consisting of uprogepant, rimegepant, and atogepant.
